(19) United States
(12) Patent Application Publication
(10) Pub. No.: US 2024/0307485 A1
(43) Pub. Date: Sep. 19, 2024

(54) WS635 USES THEREOF IN MEDICINE

(71) Applicant: WATERSTONE PHARMACEUTICALS (WUHAN) CO., LTD., Wuhan (CN)

(72) Inventors: Faming ZHANG, Wuhan (CN); Yuan SHEN, Wuhan (CN); Minglong HU, Wuhan (CN); Yao YU, Wuhan (CN); Xiaolong WANG, Wuhan (CN); Along ZHAO, Wuhan (CN)

(21) Appl. No.: 18/674,941

(22) Filed: May 27, 2024

Related U.S. Application Data
(63) Continuation of application No. PCT/CN2021/136859, filed on Dec. 9, 2021.

(51) Int. Cl. A61K 38/13; A61P 17/02

(52) U.S. Cl. CPC A61K 38/13; A61P 17/02

(57) ABSTRACT

Provided is a method of inducing or accelerating a healing process of wound in a subject in need thereof. The method includes administering to the subject a therapeutically effective amount of a compound of Formula I or a stereoisomer, a tantomer, an N-oxide, a solvate, a metabolite, a pharmaceutically acceptable salt or a prodrug thereof,

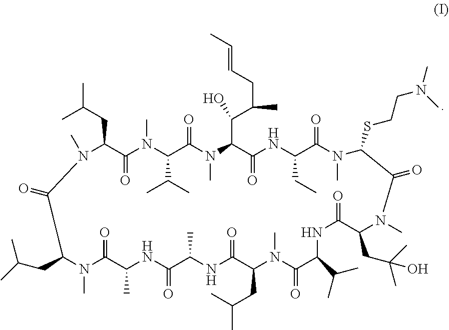 [SMILES: C/C=C/C[C@@H](C)[C@@H](O)[C@H]1C(=O)N[C@@H](CC)C(=O)N(C)[C@H](SCCN(C)C)C(=O)N(C)[C@@H](CC(C)(C)O)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

### CROSS-REFERENCE TO RELATED APPLICATIONS

This application is a continuation of International Application No. PCT/CN2021/136859, filed on Dec. 9, 2021, which is hereby incorporated by reference in its entirety.

### FIELD

The present disclosure belongs to the field of medicine. Specifically, it relates to the uses of (3S,6S,9S,12R,15S,18S,21S,24S,27R,30S,33S)-27-((2-(dimethylamino)ethyl)thio)-30-ethyl-3 3-((1R,2R,E)-1-hydroxy-2-methylhex-4-en-1-yl)-24-(2-hydroxy-2-methylpropyl)-6,9,18-triisobutyl-3,21-diisopropyl-1,4,7,10,12,15,19,25,28-nonamethyl-1,4,7,10,13,16,19,22,25,28,31-undecaazacyclotritriacontan-2,5,8,11,14,17,20,23,26,29,32-undecaone (I) (WS635, also known as SCY-635) and pharmaceutically compositions thereof in the manufacture of a medicament for promoting a healing process of wound in a patient.

### BACKGROUND

The skin forms a physical barrier, which is essential to prevent microbial invasion and maintain temperature and fluid balance. Skin wound can temporarily destroy this barrier and thus pose a huge health challenge. Wound healing is a complex process, which is characterized by the dynamic changes of the wound microenvironment recruiting and guiding different types of participating cells. The entire healing process of wound is typically divided into four consecutive overlapping stages: hemostasis, inflammation, proliferation, and remodeling. In the hemostasis phase, bleeding is controlled by sympathetic nerve-induced vasoconstriction and thrombosis. The cells of the injured tissue release alarm signals, chemokines and growth factors, recruit immune cells from the blood circulation, stimulate the proliferation of resident cells in the tissue, and cause immune cells to accumulate in the wound site. The proliferation stage is characterized by the formation of granulation tissue, which is composed of newly formed blood vessels, immune cells and fibroblasts, and allows epidermal cells to migrate to this tissue during the re-epithelialization of the wound. In the process of scar tissue formation, fibroblasts in the dermis and epidermis deposit new extracellular matrix to strengthen the repaired tissue. The skin structure and composition of different species are different, and the speed of healing is also different.

At present, most local treatments for wound healing include functional dressings (e.g., interactive dressings and bioactive dressings), biological materials (e.g., xenogeneic acellular dermal matrix and tissue engineered skin), and negative pressure treatment techniques. Among the existing oral drugs, most of the auxiliary aspects of wound care, such as pain management, infection relief, and nutrition, are extremely lacking in oral drugs that directly promote wound healing. Because oral preparations are easy to use and accelerate healing time, increasing effective wound healing oral preparations will be very beneficial to the field of wound care.

Studies have reported that compared with the control group, the open wound area of rats' skin wounds injected with growth factors is reduced. In addition, microscopic observation showed that the vascular endothelial growth factor, fibroblast growth factor and insulin growth factor increased in the wound where the growth factor was injected, which promoted the growth of epithelial cells and angiogenesis, accelerated fibrous proliferation and the deposition of type I collagen, thereby Speed up wound healing.

In another study, the results of in vitro vascular remodeling evaluation on the aorta of WT and CypD KO mice showed that the onset of CypD KO mice was earlier and the wound closure speed was significantly faster, which was related to the increased distribution of blood vessels around the wound 14 days after ear removal.

WS635 (the compound of formula (I)) is a non-immunosuppressive derivative of CsA (cyclosporin A) double-substituted at the 3 and 4 positions that binded to CypD (Cyclophilin D) to inhibit the opening of mPTP. Therefore, WS635 warrants further investigation as a novel type of mPTP inhibitor.

### SUMMARY

The present disclosure provides a method of promoting a healing process of wound in a subject in need thereof. The method includes: administering to the subject a therapeutically effective amount of a compound of Formula I or a stereoisomer, a tautomer, an N-oxide, a solvate, a metabolite, a pharmaceutically acceptable salt or a prodrug thereof,

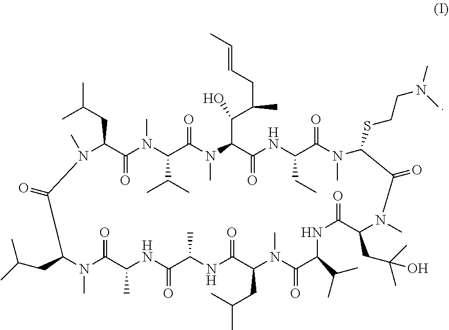 [SMILES: C/C=C/C[C@@H](C)[C@@H](O)[C@H]1C(=O)N[C@@H](CC)C(=O)N(C)[C@H](SCCN(C)C)C(=O)N(C)[C@@H](CC(C)(C)O)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

### BRIEF DESCRIPTION OF THE DRAWINGS

FIG. 1 shows that WS635 significantly promotes wound healing, and its effect is equivalent to rb-bFGF.

FIG. 2 shows that WS635 enhances wound healing rate in a time-dependent manner.

### DETAILED DESCRIPTION

### Definitions and General Terminology

Reference will now be made in detail to certain embodiments of the disclosure, examples of which are illustrated in the accompanying structures and formulas. The disclosure is intended to cover all alternatives, modifications, and equivalents which may be included within the scope of the present disclosure as defined by the claims. One skilled in the art will recognize many methods and materials similar or equivalent to those described herein, which could be used in the practice of the present disclosure. The present disclosure is in no way limited to the methods and materials described herein. In the event that one or more of the incorporated literature, patents, and similar materials differs from or contradicts this application, including but not limited to defined terms, term usage, described techniques, or the like, this application controls.

It is further appreciated that certain features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable sub-combination.

Unless defined otherwise, all scientific and technical terms used herein have the same meaning as is commonly understood by one skilled in the art to which this disclosure belongs. All patents and publications referred to herein are incorporated by reference in their entirety.

The grammatical articles “a”, “an” and “the”, as used herein, are intended to include “at least one” or “one or more” unless otherwise indicated herein or clearly contradicted by the context. Thus, the articles used herein refer to one or more than one (i.e. at least one) of the grammatical objects of the article. For example, “an embodiment” refers to one or more embodiments.

The term “comprise” or “include” is an open expression, it means that the contents disclosed herein are included, without excluding other contents.

The term “pharmaceutically acceptable” as used herein, refers to those compounds, materials, compositions, and/or dosage forms which are, within the scope of sound medical judgment, suitable for use in contact with the tissues of patients without excessive toxicity, irritation, allergic response, or other problem or complication commensurate with a reasonable benefit/risk ratio, and are effective for their intended use.

The term “prodrug” refers to a compound that is transformed in vivo into a compound of Formula (I). Such a transformation can be affected, for example, by hydrolysis of the prodrug form in blood or enzymatic transformation to the parent form in blood or tissue. Prodrugs of the compounds disclosed herein may be, for example, esters. Some common esters which have been utilized as prodrugs are phenyl esters, aliphatic (C1-24) esters, acyloxymethyl esters, carbonates, carbamates and amino acid esters. For example, a compound disclosed herein that contains a hydroxy group may be acylated at this position in its prodrug form. Other prodrug forms include phosphates, such as, those phosphate compounds derived from the phosphonation of a hydroxy group on the parent compound. A thorough discussion of prodrugs is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987, J. Rautio et al., Prodrugs: Design and Clinical Applications, Nature Review Drug Discovery, 2008, 7, 255-270, and S. J. Hecker et al., Prodrugs of Phosphates and Phosphonates, Journal of Medicinal Chemistry, 2008, 51, 2328-2345, each of which is incorporated herein by reference.

A “metabolite” is a product produced through metabolism in the body of a specified compound or salt thereof. The metabolites of a compound may be identified using routine techniques known in the art and their activities determined using tests such as those described herein. Such products may result for example from oxidation, reduction, hydrolysis, amidation, deamidation, esterification, deesterification, enzyme cleavage, and the like, of the administered compound. Accordingly, the disclosure includes metabolites of compounds disclosed herein, including metabolites produced by contacting a compound disclosed herein with a mammal for a sufficient time period.

A “pharmaceutically acceptable salts” refers to organic or inorganic salts of a compound disclosed herein. Pharmaceutically acceptable salts are well known in the art. For example, S. M. Berge et al., describe pharmaceutically acceptable salts in detail in J. Pharmaceutical Sciences, 1977, 66: 1-19, which is incorporated herein by reference. Some non-limiting examples of pharmaceutically acceptable and nontoxic salts include salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid and malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, laurylsulfate, malate, methanesulfonate, 2-naphthalenesulfonate, nicotinate, nitrate, oleate, palmitate, pamoate, pectinate, persulfate, 3-phenylpropionate, picrate, pivalate, propionate, stearate, thiocyanate, p-toluenesulfonate, undecanoate, valerate, and the like. Salts derived from appropriate bases include alkali metal, alkaline earth metal, ammonium and N+(C1-4 alkyl)4 salts. This disclosure also envisions the quaternization of any basic nitrogen-containing groups of the compounds disclosed herein. Water or oil soluble or dispersible products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, C1-8 sulfonate or aryl sulfonate.

The term “solvate” refers to an association or complex of one or more solvent molecules and a compound disclosed herein. Examples of solvents that form solvates include, but are not limited to, water, isopropanol, ethanol, methanol, DMSO, ethyl acetate, acetic acid, ethanolamine and the mixture thereof. The term “hydrate” refers to the complex where the solvent molecule is water.

The term “hydrate” can be used when said solvent is water. In an embodiment, one water molecule is associated with one molecule of the compounds disclosed herein, such as a hydrate. In another embodiment, more than one water molecule may be associated with one molecule of the compounds disclosed herein, such as a dihydrate. In still another embodiment, less than one water molecule may be associated with one molecule of the compounds disclosed herein, such as a hemihydrate. Furthermore, all the solvates of the disclosure retain the biological effectiveness of the non-hydrate form of the compounds disclosed herein.

As used herein, the term “therapeutically effective amount” or “therapeutically effective dose” refers to the amount of compound disclosed herein that can elicit the biological or medical response (such as reducing or inhibiting the activity of an enzyme or protein, or improving symptoms, lessening disorders, slowing or delaying the development of diseases and the like).

### Use of the Compounds and Pharmaceutical Compositions

In the research and development process, the Applicant surprisingly found that WS635 could significantly promote angiogenesis in rats. The Applicant further investigated that the WS635 could be used to promoting a healing process of wound. WS635 as the active ingredient of the healing wounds therapeutic agents has less toxic effects, which the NOAEL (No Observed Adverse Effect Level) dose was 80 mg/kg/day for toxicity test of repeated oral administration for 9 days in rats. Moreover, WS635 has better stability, pharmacokinetics etc., and WS635 has already been shown to be more effective in healing wounds.

The present disclosure provides a method of promoting a healing process of wound in a subject in need thereof. The method includes: administering to the subject a therapeutically effective amount of a compound of Formula I or a stereoisomer, a tautomer, an N-oxide, a solvate, a metabolite, a pharmaceutically acceptable salt or a prodrug thereof,

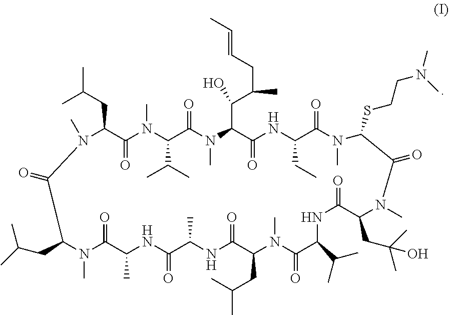 [SMILES: C/C=C/C[C@@H](C)[C@@H](O)[C@H]1C(=O)N[C@@H](CC)C(=O)N(C)[C@H](SCCN(C)C)C(=O)N(C)[C@@H](CC(C)(C)O)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

In the research and development process, the Applicant surprisingly found that WS635 could promote angiogenesis in rats. According to the examples of present disclosure, the said compound of formula (I) can promote angiogenesis and promote a healing wound.

In an embodiment, the wound is a surgery-induced wound.

In an embodiment, the wound is a skin wound.

In an embodiment, the compound is administered to the subject after the wound formed.

In an embodiment, the compound is administered to the subject within 14 days after the wound formed.

In an embodiment, the compound is administered to the subject within 7 days after the wound formed.

In an embodiment, the compound is administered at a daily dose of less than about 1000 mg.

In an embodiment, the compound is administered at a daily dose of between about 30 to about 1000 mg.

In an embodiment, the compound is administered at a daily dose of between about 500 to about 700 mg.

In an embodiment, the compound is administered 1 time per day.

In an embodiment, the compound is administered 1 time per day as a single dosage.

In an embodiment, the compound is administered orally.

In an embodiment, the compound is administered in a form of tablet, capsule or injection.

In an embodiment, the compound is administered in combination with one or more other agent used for inducing or accelerating a healing process of wound other than the compound of Formula I.

In another aspect, provided herein is use of a compound of Formula I or a stereoisomer, a tautomer, an N-oxide, a solvate, a metabolite, a pharmaceutically acceptable salt or a prodrug thereof in promoting a healing process of wound in a subject in need thereof,

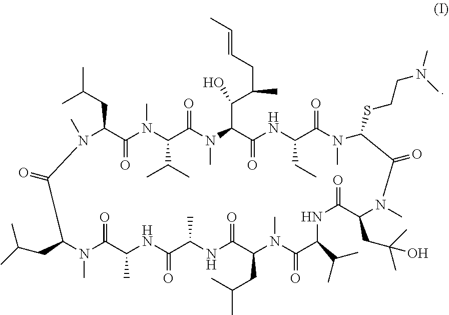 [SMILES: C/C=C/C[C@@H](C)[C@@H](O)[C@H]1C(=O)N[C@@H](CC)C(=O)N(C)[C@H](SCCN(C)C)C(=O)N(C)[C@@H](CC(C)(C)O)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

In an embodiment, the wound is a surgery-induced wound.

In an embodiment, the wound is a skin wound.

In an embodiment, the compound is administered to the subject after the wound formed.

In an embodiment, the compound is administered to the subject within 14 days after the wound formed.

In an embodiment, the compound is administered to the subject within 7 days after the wound formed.

In an embodiment, the compound is administered at a daily dose of less than about 1000 mg.

In an embodiment, the compound is administered at a daily dose of between about 30 to about 1000 mg.

In an embodiment, the compound is administered at a daily dose of between about 500 to about 700 mg.

In an embodiment, the compound is administered 1 time per day.

In an embodiment, the compound is administered 1 time per day as a single dosage.

In an embodiment, the compound is administered orally.

In an embodiment, the compound is administered in a form of tablet, capsule or injection.

In an embodiment, the compound is administered in combination with one or more other agent used for inducing or accelerating a healing process of wound other than the compound of Formula I.

Any embodiment disclosed herein can be combined with other embodiments as long as they are not contradictory to one another, even though the embodiments are described under different aspects of the disclosure. In addition, any technical feature in an embodiment can be applied to the corresponding technical feature in other embodiments as long as they are not contradictory to one another, even though the embodiments are described under different aspects of the disclosure.

The foregoing merely summarizes certain aspects disclosed herein and is not intended to be limiting in nature. These aspects and other aspects and embodiments are described more fully below.

An “effective amount”, “a therapeutically effective amount” or “effective dose” of the compound, or a stereoisomer, a tautomer, an N-oxide, a solvate, a metabolite, a pharmaceutically acceptable salt or a prodrug thereof or pharmaceutically acceptable composition disclosed herein is an amount that is effective promoting healing wound. The complex and pharmaceutically acceptable compositions are effectively administered in a fairly wide dose range. For example, the daily dose is from about 30 to about 1000 mg per person, the compounds or pharmaceutically acceptable compositions can be administered in a single dose or in several divided doses a day. The compound and compositions, according to the method disclosed herein, may be administered using any amount and any route of administration which is effective for promoting healing wound. The exact amount required will vary from subject to subject, depending on the species, age, and general condition of the subject, the severity of the infection, the particular agent, its mode of administration, and the like.

In some embodiments, the compound described herein can be administered to a subject after the wound formed, for example within 14 days after the wound is formed. The compound described herein can be administered at a daily dose of less than about 1000 mg, for example, at a daily dose of between about 30 to about 1000 mg or at a daily dose of between about 500 to about 700 mg. The compound can be administered 1 time per day or can be administered 1 time per day as a single dosage.

Besides being useful for human treatment, WS635 and the compositions thereof are also useful for veterinary treatment of animals such as companion animals, exotic animals and mammals of farm animals. In other embodiments, the animals disclosed herein include horses, dogs, and cats. As used herein, the compounds disclosed herein include the pharmaceutically acceptable derivatives thereof.

### EXAMPLES

### Materials and Methods

Eighteen 6-week-old male SD rats were randomly divided into 3 groups: model group, test article group and positive control group, with 6 rats in each group. Skin wound surgery was performed after anesthesia, and oral administration was started 2 h after skin wound formation. All rats were raised in a single cage. All rats were raised in a single cage. The surgical details were as follows: All animals were anesthetized with 10% chloral hydrate solution (3 mL/kg) by intraperitoneal injection, their backs were depatured with pet shaving knives, and a circular mark with a diameter of 2 cm was made. After the skin was disinfected with alcohol, the full layer of skin was removed with surgical scissors along the marking line, and the wound surface was pressed to stop bleeding. The model group was orally given 10% DMSO corn oil, the test group was orally given WS635 (corn oil dissolved in 10% DMSO) at 52.8 mg/kg, and the positive control group was given 262.5 IU/cm2 (62.5 μL/cm²) recombinant bovine basic fibroblast growth factor (rb-bFGF) external solution, once a day for 7 days. Digital photos were taken of the wound of each rat under the same conditions. The wound was recorded with plastic transparent paper, and the wound area was scanned by image J software after the wound was recorded with plastic transparent paper. Wound healing rate was calculated according to wound area (%)=(area after administration−area before administration)/area before administration 100%.

### Result

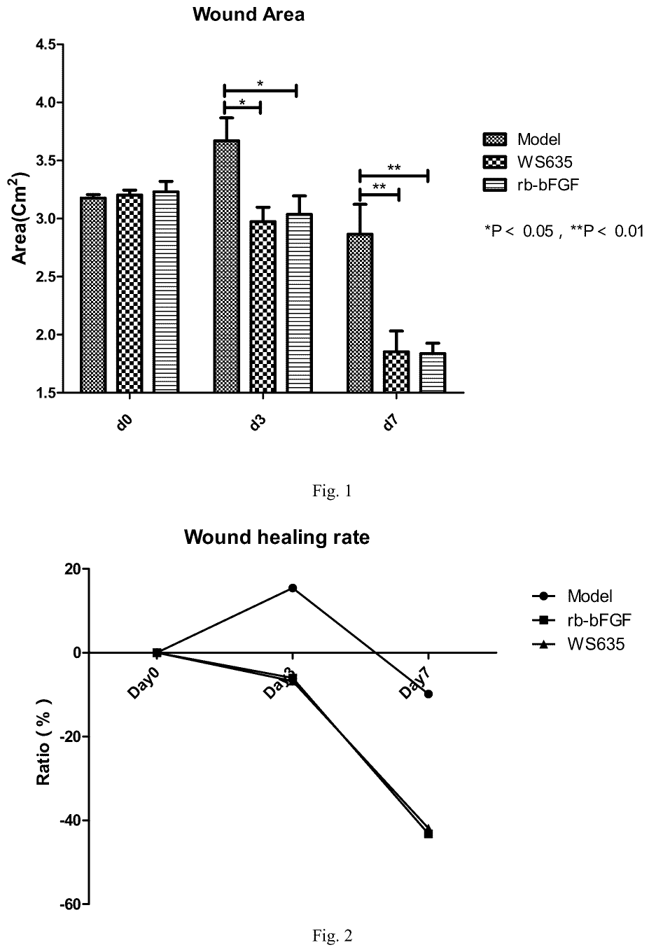

Compared with the model group, the wound area of WS635 and positive control (rb-bFGF) decreased significantly on Day 3 and Day 7 (P<0.05, P<0.01). WS635 and rb-bFGF had no significant difference (P>0.05). Within the administration cycle, the wound healing rate of WS635 and rb-bFGF groups increased with the extension of administration time. The results showed that oral administration of WS635 can significantly promote wound healing in the wound healing model of SD rats. The results are shown in FIG. 1 and FIG. 2.

What is claimed is:

1. A method of promoting a healing process of wound in a subject in need thereof, the method comprising:
administering to the subject a therapeutically effective amount of a compound of Formula I or a stereoisomer, a tautomer, an N-oxide, a solvate, a metabolite, a pharmaceutically acceptable salt or a prodrug thereof,

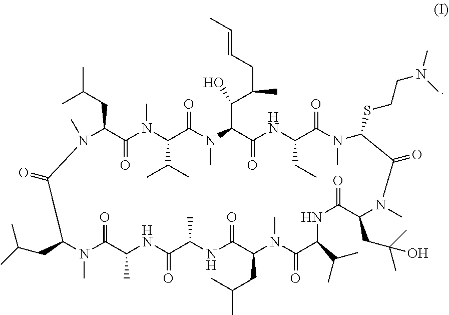 [SMILES: C/C=C/C[C@@H](C)[C@@H](O)[C@H]1C(=O)N[C@@H](CC)C(=O)N(C)[C@H](SCCN(C)C)C(=O)N(C)[C@@H](CC(C)(C)O)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

2. The method of claim 1, wherein the wound is a surgery-induced wound.

3. The method of claim 1, wherein the wound is a skin wound.

4. The method of claim 1, wherein the compound is administered to the subject after the wound formed.

5. The method of claim 1, wherein the compound is administered to the subject within 14 days after the wound formed.

6. The method of claim 1, wherein the compound is administered to the subject within 7 days after the wound formed.

7. The method of claim 1, wherein the compound is administered at a daily dose of less than about 1000 mg.

8. The method of claim 1, wherein the compound is administered at a daily dose of between about 30 to about 1000 mg.

9. The method of claim 1, wherein the compound is administered at a daily dose ranging from about 500 mg to about 700 mg.

10. The method of claim 1, wherein the compound is administered 1 time per day.

11. The method of claim 1, wherein the compound is administered 1 time per day as a single dosage.

12. The method of claim 1, wherein the compound is administered orally.

13. The method of claim 1, wherein the compound is administered in a form of tablet, capsule or injection.

14. The method of claim 1, wherein the compound is administered in combination with one or more other agent used for inducing or accelerating a healing process of wound other than the compound of Formula I.
